(19) United States
(12) Patent Application Publication
(10) Pub. No.: US 2006/0216250 A1
(43) Pub. Date: Sep. 28, 2006

(54) Nail polish compositions comprising a water or alcohol soluble polyamide

(76) Inventors: Thomas M. Schultz, Randolph, NJ (US); Rolando Umali Pagilagan, Parkersburg, WV (US)

Correspondence Address: E I DU PONT DE NEMOURS AND COMPANY;LEGAL PATENT RECORDS CENTER, BARLEY MILL PLAZA 25/1128, 4417 LANCASTER PIKE, WILMINGTON, DE, 19805, US

(21) Appl. No.: 11/232,637

(22) Filed: Sep. 22, 2005

Related U.S. Application Data
(60) Provisional application No. 60/620,474, filed on Oct. 20, 2004.

(51) Int. Cl. A61K 8/88

(52) U.S. Cl. 424/59; 424/61

(57) ABSTRACT

Nail polish compositions comprising a water or alcohol soluble polyamide are described. The water or alcohol soluble polyamides are prepared from the reaction of adipic acid and certain ether diamines having a molecular weight of 148 to 396. The nail polish compositions can be either non-aqueous or aqueous, and can be in the form of color enamels, nail varnishes, nail lacquers, clear base coats, topcoats, and nail hardeners.

### FIELD OF THE INVENTION

The invention relates to the field of personal care products. More specifically, the invention relates to nail polish compositions comprising a water or alcohol soluble polyamide.

### BACKGROUND OF THE INVENTION

Typical nail polish compositions have as their base a mixture of organic solvents containing nitrocellulose as the primary film-forming agent, an aryl-sulfamide formaldehyde resin, or an alkyd resin, and a plasticizing agent. These components are quite flammable and toxic. In particular, nitrocellulose is highly flammable, and presents safety hazards for its manufacture, transportation, and storage. Additionally, the solvents used to dissolve nitrocellulose are also flammable. Therefore, alternative film-forming agents have been sought to supplement nitrocellulose, so that smaller amounts are required, or to replace nitrocellulose entirely. Examples of these alternative film-forming agents include acrylic and polyester resins.

Aqueous nail polish compositions have also been developed because of the aforementioned problems with conventional, organic solvent-based compositions. The aqueous compositions generally contain polyurethane, polyurethane copolymers, acrylic polymers or acrylic copolymers in water or alcohol-water mixtures. These aqueous compositions mitigate much of the problem associated with non-aqueous compositions; however, they typically are slow drying. Therefore, faster setting film-forming agents for use in aqueous nail polish compositions would be highly desirable.

Polyamides are well known for their desirable properties such as strength, toughness, abrasion resistance, lubricity, and chemical resistance. They also have very high gloss from a physical appearance standpoint. Moreover, polyamides are chemically similar to proteins and might be expected to assist with the wetting or spreading of formulations containing them along the surface of nails. However, polyamides in solution form have not been widely used in nail polish compositions because of their poor solubility.

Nylon (polyamide) fibers have been used in nail polish compositions for some time. However, the fibers are soft and flexible, and the adhesion between the fibers and the nail polish resin is low (Sheard et al., U.S. Pat. No. 5,330,750).

Blin et al. (U.S. Patent Application Publication No. 2002/0192168) describe gel or stick nail polish compositions containing a polymer, which may be a polyamide. The polyamides described in that disclosure are formed from the polycondensation between a dicarboxylic acid, which is a dimer resulting from a fatty acid such as oleic, linoleic, or linolenic acid, with a diamine such as ethylenediamine, hexylenediamine, or hexamethylenediamine. The polyamides have a molecular weight of 1,000 to 100,000 and are not soluble in water or alcohol.

The use of water or alcohol soluble polyamides in personal care products is described by Pagilagan in U.S. Patent Application Publication No. 2003/0235551. However, the use of these polyamides in nail polish compositions is not described in that disclosure.

Therefore, the need exists for new film-forming agents to be used in both non-aqueous and aqueous nail polish compositions.

Applicants have addressed the stated need by discovering that water or alcohol soluble polyamides function as effective film-forming agents in both non-aqueous and aqueous nail polish compositions. In non-aqueous compositions, the use of the water or alcohol soluble polyamide may serve to reduce the amount of low vapor pressure organic solvents and the amount of nitrocellulose, formaldehyde, or polyacrylates required, resulting in a safer, more environmentally friendly composition.

### SUMMARY OF THE INVENTION

The invention provides for a nail polish composition comprising: an effective amount of a water or alcohol soluble polyamide having a solubility of at least 0.5 weight percent in water or in a C₁ to C₃ alcohol and derived from the reaction of adipic acid and ether diamines with a molecular weight of 148 to 396 and represented by the general formulas: H₂N—R₁—O—R₂—O—R₁—NH₂, wherein R₁ and R₂ are either —CH₂—CH₂— or —CH₂—CH₂—CH₂—; or H₂N—R₁(—O—CH₂—CH₂)ₓO—R₁—NH₂, wherein R₁ is either —CH₂—CH₂— or —CH₂—CH₂—CH₂— and X has an average value of 2 to 6; and mixtures thereof.

The composition may also comprise at least one cosmetic component selected from the group consisting of solvents, film-forming substances, plasticizers, dyes, pigments, iridescent substances, suspending agents, coalescing agents, thickeners, surfactants, wetting agents, slip aids, preservatives, vitamins, UV absorbing organic sunscreens, UV scattering inorganic sunscreens, natural proteins, and recombinant proteins.

The effective amount of water or alcohol soluble polyamide is from about 0.1% to about 40% by weight relative to the total weight of the composition, preferably about 1% to about 40% by weight relative to the total weight of the composition.

The composition may be. in the form of a color, enamel, nail varnish, nail lacquer, clear base coat, topcoat, or nail hardener.

The invention also provides a method for forming a coating of nail polish film comprising a water or alcohol soluble polyamide on nails comprising applying to the nails the composition described above and allowing the composition to dry on the nails to form a film.

### DETAILED DESCRIPTION OF THE INVENTION

Applicants specifically incorporate the entire content of all cited references in this disclosure. Further, when an amount, concentration, or other value or parameter is given as either a range, preferred range, or a list of upper preferable values and lower preferable values, this is to be understood as specifically disclosing all ranges formed from any pair of any upper range limit or preferred value and any lower range limit or preferred value, regardless of whether ranges are separately disclosed. Where a range of numerical values is recited herein, unless otherwise stated, the range is intended to include the endpoints thereof, and all integers and fractions within the range. It is not intended that the scope of the invention be limited to the specific values recited when defining a range.

The invention provides nail polish compositions comprising a water or alcohol soluble polyamide. The water or alcohol soluble polyamide functions as an effective film-forming agent in both non-aqueous and aqueous nail polish compositions, and may also function as a plasticizer. The water or alcohol soluble polyamides are useful in nail polish compositions, including but not limited to color enamels, nail varnishes, nail lacquers, clear base coats, topcoats, and nail hardeners.

The following definitions are used herein and should be referred to for interpretation of the claims and the specification.

The term “nail polish” as used herein, is a comprehensive term used to describe a composition useful for providing aesthetic, therapeutic, or prophylactic benefits to the nail. The nail polish compositions of the invention include, but are not limited, to color enamels, nail varnishes, nail lacquers, clear base coats, topcoats, and nail hardeners.

The term “nails” as used herein refers to mammalian nails, preferably human fingernails and toenails.

The term “natural protein” is used to describe a protein that is isolated from its native animal, microbial, bacterial, yeast or plant source.

The term “recombinant protein” is used to describe a protein that is produced by a non-native organism using genetic engineering techniques. Both natural proteins and recombinant proteins can exist in their native sequences or in sequences modified by genetic engineering.

The term “water or alcohol soluble polyamide” means a polyamide having a solubility of at least 0.5 weight % in water or in a C₁ to C₃ alcohol. Thus, a polyamide can be soluble in water, can be soluble in alcohol, or can be soluble in both water and alcohol so long as the polyamide has a solubility of at least 0.5 weight % in at least one of the solvents.

Water or Alcohol Soluble Polyamides:

The water or alcohol soluble polyamides of the invention are described by Pagilagan, supra, which is incorporated herein by reference. These polyamides have a solubility of at least 0.5 weight percent in water or in a C₁ to C₃ alcohol and are derived from the reaction of adipic acid and ether diamines with a molecular weight of 148 to 396 and represented by the general formulas: H₂N—R₁—O—R₂—O—R₁—NH₂, wherein R₁ and R₂ are either —CH₂—CH₂— or —CH₂—CH₂—CH₂—; H₂N—R₁(—O—CH₂—CH₂)ₓO—R₁—NH₂, wherein R₁ is either —CH₂—CH₂— or —CH₂—CH₂—CH₂— and X has an average value of 2 to 6; and mixtures thereof.

Copolyamides of the above polyamide with other polyamide-forming comonomers can also be used herein. These other polyamide-forming comonomers may be incorporated into the compositions described herein provided these comonomers do not adversely affect the water or alcohol solubility of the resulting polyamide. These added comonomers may include other polyamide forming comonomers such as lactates, polyether diamines, polyether diacids, alkylene diamines, and alkylene dicarboxylic acids. The solubility in water or alcohol of these polyamides is influenced not only by the amount of the polyetherdiamines and the nature of the dicarboxylic acids but by the molecular weight as well. Polyethylene glycol diamines and diacids are of particular interest as a polyamide-forming comonomer with the polyamides described above.

The water or alcohol soluble polyamides of the invention may be prepared using standard nylon polymerization methods that are well-known in the art (See Kohan, M. I. , Nylon Plastics Handbook, Hansen/Gardner Publications, Inc. [1995] pages 17-20 & 34-45). Specific procedures are given in Examples 1-10.

Nail Polish Compositions:

The nail polish compositions of the invention include, but are not limited to, color enamels, nail varnishes, nail lacquers, clear base coats, topcoats, and nail hardeners. The nail polish compositions comprise an effective amount of a water or alcohol soluble polyamide, as described supra, or mixtures thereof, in a cosmetically acceptable medium. An effective amount of a water or alcohol soluble polyamide is herein defined as a proportion of about 0.1% to about 40%, preferably from about 1% to about 40% by weight of the total composition.

Components of a cosmetically acceptable medium for nail polish compositions are well known in the art. The nail polish composition typically contains a solvent, a film-forming substance, and a plasticizer. Additionally, the nail polish composition may comprise various other adjuvants, such as dyes, pigments, suspending agents, coalescing agents, thickeners, surfactants, wetting agents, slip aids, preservatives, vitamins, UV absorbing organic sunscreens, UV scattering inorganic sunscreens, and natural and recombinant proteins.

In one embodiment, the solvent is a mixture of various volatile organic solvents. The solvent system for the nail polish is generally present in a proportion ranging from about 55 to about 90% by weight relative to the total weight of the composition. Suitable organic solvents include, but are not limited to, acetone, ethyl acetate, butyl acetate, 2-methoxyethyl acetate, methyl ethyl ketone, methyl isobutyl ketone, methyl acetate, amyl acetate, isopropyl acetate, and mixtures thereof. The nail polish composition may also contain a diluent, which is preferably a saturated, linear or branched hydrocarbon, such as hexane or octane, or alternatively an aromatic hydrocarbon such as toluene or xylene, in a proportion of from about 10 to about 35% by weight relative to the total weight of the composition. The solvent system may also include other volatile solvents such as ethanol, n-butanol, n-propanol, isopropanol, and mixtures thereof.

In another embodiment, the solvent is aqueous-based consisting of water or a mixture of water with a water-miscible organic solvent such as acetone, ethanol, n-butanol, n-propanol, isopropanol and mixtures thereof.

In addition to the water or alcohol soluble polyamide, the composition according to the invention may also comprise film-forming substances. The film-forming substance is generally present in a proportion of about 5 to about 35% by weight relative to the total weight of the composition. Suitable film-forming substances include, but are not limited to, nitrocelluloses; polyvinyl derivatives such as polyvinyl butyrate; cellulose derivatives other than nitrocellulose; acrylic polymers or copolymers; acrylic, styrene, acrylate-styrene and vinyl resins; vinyl copolymers; polyester polymers; arylsulphonamide resins; alkyd resins; polyurethanes; polyurethane copolymers; and mixtures thereof.

The nail polish compositions of the invention may also comprise plasticizers, which are generally present in a proportion of about 5 to about 20% by weight relative to the total weight of the composition. The plasticizers allow the flexibility of the film to be adjusted without reducing its strength or its physical force. Suitable plasticizers include, but are not limited to, tricresyl phosphate, benzyl benzoate, tributyl phosphate, butyl acetyl ricinoleate, triethyl citrate, tributyl acetyl citrate, dibutyl phthalate, camphor and mixtures thereof.

The nail polish compositions of the invention may be either colorless or colored. When they are colored, they then contain pigments and/or dyes, such as those disclosed in the CTFA Cosmetic Ingredient Handbook, 9^{th} edition (2002). The pigments and dyes are typically present in a proportion of about 0.01% to about 5% relative to the total weight of the nail polish composition. Suitable dyes and pigments include, but are not limited to, dyes, such as D&C Red Nos. 21, 27; D&C Red Orange Nos. 5, 10; organic pigments such as D&C Red Nos. 8, 10, 11, 12, 13, 30 and 36; the barium lakes of D&C Red Nos. 6, 9 and 12; the calcium lakes of D&C Red Nos. 7, 11, 31 and 34; the strontium lake of D&C Red No. 30, D&C Orange No. 17, and D&C Blue No. 6; inorganic pigments such as iron oxides, titanium dioxide, and bismuth oxychloride; and iridescent substances such as guanine.

The nail polish compositions of the invention may also comprise suspending agents to prevent settling of particulates. The suspending agents are typically present in a proportion of about 0.05% to about 5% by weight relative to the total weight of the composition. Suitable suspending agents include, but are not limited to, modified montmorillonite clays, such as Bentone® 27, Bentone® 34, or Bentone® 38; modified smectite, hectorite, and bentonite, and mixtures thereof.

The nail polish compositions of the invention may also comprise coalescing agents to enhance film formation, particularly wherein the film-forming agent is water-borne. The coalescing agents are typically present in a proportion of about 0.1% to about 10% by weight relative to the total weight of the composition. Such coalescing aides are known in the art and are typically glycol ethers or glycol ether esters, such as C₁ to C₁₀ straight or branched chain alkyl glycol alkyl ethers, C₁ to C₁₀ straight or branched chain alkyl ether acetates, di-C₁ to C₁₀ straight or branched chain alkyl ether acetates, and C₁ to C₁₀ alkyl glycol phenyl ethers. Suitable coalescing agents include, but are not limited to, ethylene glycol ethers, diethylene glycol ethers, triethylene glycol ethers, propylene glycol ethers, dipropylene glycol ethers, terpenes, camphor, methyl cellusolve, butyl cellusolve™ (ethylene glycol monobutyl ether), and methyl carbitol.

The nail polish compositions of the invention may also comprise thickeners to achieve the desired rheology and application properties. Thickeners are generally utilized wherein the nail polish composition comprises a water-borne film former or at least 4% water. The thickeners are typically present in a proportion of about 0.1% to about 10% by weight relative to the total weight of the composition. Suitable thickeners include, but are not limited to, hydroxyethyl cellulose, hydroxypropyl cellulose, methyl cellulose, guar gum, gum arabic, xantham gum, and carbomer polymers.

The nail polish compositions of the invention may also comprise surfactants or wetting agents, typically at about 0.001 to about 18%, preferably, at about 0.005 to about 15% by weight of the total composition. The terms “surfactants” and “wetting agents” as used herein refer to surface-active agents which, when added to water, cause the active ingredient to penetrate more easily into, or spread on the surface of another material, by reducing the surface tension of the water at the water-air or water-oil interface. By “surface active agent” is meant any compound that reduces surface tension when dissolved in water or water solutions. The selection of a surfactant for this purpose presents a wide range of possibilities known in the art. Suitable surfactants include, but are not limited to, the following:

(1) anionic surfactants, such as metallic or alkanolamine salts of fatty acids, for example sodium laurate and triethanolamine oleate; alkyl benzene sulfones, for example triethanolamine dodecyl benzene sulfonate; alkyl sulfates, for example sodium lauryl sulfate; alkyl ether sulfates, for example sodium lauryl ether sulfate (2 to 8 EO); sulfosuccinates, for example sodium dioctyl sulfonsuccinate; monoglyceride sulfates, for example sodium glyceryl monostearate monosulfate; isothionates, for example sodium isothionate; methyl taurides, for example Igepon T; acylsarcosinates, for example sodium myristyl sarcosinate; acyl peptides, for example Maypons and lamepons; acyl lactylates; polyalkoxylated ether glycollates, for example trideceth-7 carboxylic acid; phosphates, for example sodium dilauryl phosphate.

(2) cationic surfactants, such as amine salts, for example sapamin hydrochloride; quaternary ammonium salts, for example Quaternium 5, Quaternium 31 and Quaternium 18;

(3) amphoteric surfactants, such as imidazol compounds, for example Miranol; N-alkyl amino acids, such as sodium cocaminopropionate and asparagine derivatives; betaines, for example cocamidopropylebetaine;

(4) nonionic surfactants, such as fatty acid alkanolamides, for example oleic ethanolamide; esters or polyalcohols, for example Span; polyglycerol esters, for example those esterified with C₁₂₋₁₈ fatty acids and one or several OH groups; polyalkoxylated derivatives, for example polyoxy:polyoxyethylene stearate (available, for example, from McIntyre Co.); ethers, for example polyoxyethylene lauryl ether (available, for example, from Stepan Co., Northfield, Ill., as Stepanol® ES); ester ethers, for example Tween; amine oxides, for example coconut and dodecyl dimethyl amine oxides;

(5) water-soluble fluorinated surfactants, such as for example CₙF₂ₙ₊₁CH₂CH₂O(CH₂CH₂O)ₓH, wherein n=3 to 8, x=2 to 100, sold under the trade name ZONYL® FSN and ZONYL® FSN 100 by E.I. du Pont de Nemours and Co. (Wilmington, Del.); CₙF₂ₙ₊₁CH₂CH₂SCH₂CO₂Li, wherein n=3 to 8, sold under the trade name ZONYL® FSA by E.I. du Pont de Nemours and Co.; (CₙF₂ₙ₊₁CH₂CH₂O)₁₋₂P(O)(ONH₄)₂₋₁, wherein n=3 to 8, sold under the trade name ZONYL® FSP by E.I. du Pont de Nemours and Co.; and (CₙF₂ₙ₊₁CH₂CH₂O)₁₋₂P(O)(OH)₂₋₁, wherein n=3 to 8, sold under the trade name ZONYL® UR by E.I. du Pont de Nemours and Co.

Mixtures of two or more of the above surfactants can be employed in the compositions according to the invention.

The nail polish compositions of the invention may also comprise slip aids to improve surface friction, water resistance, abrasion resistance, and mechanical properties. The slip aids are typically present in a proportion of about 0.1% to about 10% by weight relative to the total weight of the composition. Suitable slip aids include, but are not limited to, wax additives, such as beeswax, carob, candelilla, paraffin waxes, polyethylene waxes, polypropylene waxes, polytetrafluoroethylene, and silicones, such as copolymers of polyether and polysiloxane.

The nail polish compositions of the invention may also comprise preservatives to prevent, inhibit, or retard microbial growth. The preservatives are typically present in a proportion of about 1% to about 10% by weight relative to the total weight of the composition. Suitable preservatives are well known in the art and include, but are not limited to, benzoic acid, benzyl alcohol, benzylhemiformal, benzylparaben, 5-bromo-5-nitro-1,3-dioxane, 2-bromo-2-nitropropane-1,3-diol, butyl paraben, phenoxyethanol, methyl paraben, ethyl paraben, propyl paraben, diazolidinyl urea, calcium benzoate, calcium propionate, captan, chlorhexidine diacetate, chlorhexidine digluconate, chlorhexidine dihydrochloride, chloroacetarnide, chlorobutanol, p-chloro-m-cresol, chlorophene, chlorothymol, chloroxylenol, m-cresol, o-cresol, DEDM Hydantoin, DEDM Hydantoin dilaurate, dehydroacetic acid, diazolidinyl urea, dibromopropamidine diisethionate, DMDM Hydantoin, Phenonip®, Kathon®, and all of those disclosed on pages 570 to 571 of the CTFA Cosmetic Ingredient Handbook, Second Edition, 1992, which is hereby incorporated by reference.

The nail polish compositions of the invention may also comprise vitamins and/or coenzymes, preferably at about 0.001 to about 10%, more preferably at about 0.01% to about 8%, most preferably at about 0.05% to about 5% by weight of the total composition. Suitable vitamins include, but are not limited to, ascorbic acid and derivatives thereof; the B vitamins, such as thiamine, riboflavin, pyridoxin, and the like; vitamin A and derivatives thereof; vitamin E and derivatives thereof; vitamin D; and vitamin K; as well as coenzymes such as thiamine pyrophosphate, flavin adenine dinucleotide, folic acid, pyridoxal phosphate, tetrahydrofolic acid, and the like.

The nail polish compositions of the invention may also comprise UV absorbing organic sunscreens, preferably at about 0.001 to about 20%, more preferably at about 0.01 to about 10%, most preferably at about 0.05 to about 8% by weight of the total composition. UV absorbing organic sunscreens are herein defined as organic chemicals that absorb ultraviolet light of wavelengths between 290 and 329 nm. Suitable UV absorbing organic sunscreens include, but are not limited to, para-aminobenzoic acid, ethyl para-aminobenzoate, amyl para-aminobenzoate, octyl para-aminobenzoate, ethylene glycol salicylate, phenyl salicylate, octyl salicylate, benzyl salicylate, butylphenyl salicylate, homomenthyl salicylate, benzyl cinnamate, 2-ethoxyethyl para-methoxycinnamate (such as Parsol® available from Givaudan-Roure Co.), octyl para-methoxycinnamate, glyceryl mono(2-ethylhexanoate) dipara-methoxycinnamate, isopropyl para-methoxycinnamate, diisopropyl-diisopropylcinnamic acid ester mixtures, urocanic acid, ethyl urocanate, hydroxymethoxybenzophenone, hydroxymethoxybenzophenonesulfonic acid and salts thereof, dihydroxymethoxybenzophenone, sodium dihydroxymethoxybenzophenonedisulfonate, dihydroxybenzophenone, tetrahydroxybenzophenone, 4-tert-butyl-4′-methoxydibenzoylmethane, 2,4,6-trianilino-p-(carbo-2′-ethylhexyl-1′-oxy)-1,3,5-triazine, and 2-(2-hydroxy-5-methylphenyl)benzotriazole.

UV scattering inorganic sunscreen materials, such as inorganic pigments and metal oxides, including but not limited to, oxides of titanium (such as SunSmart available from Cognis Corp., Duesseldorf, Germany), zinc, and iron, may also be incorporated into the compositions of the invention. UV scattering inorganic sunscreens are herein defined as inorganic substances that scatter ultraviolet light of wavelengths between 210 and 280 nm. These UV scattering inorganic sunscreens may be used in the personal care compositions of this invention at concentrations of preferably about 0.001 to about 40%, more preferably at about 0.01 to about 10%, most preferably at about 0.05 to about 8% by weight of the total composition.

The nail polish compositions of the invention may also comprise natural or recombinant proteins, typically at about 0.001 to about 60% by weight, preferably from about 0.01 to about 20% by weight relative to the total weight of the composition. Suitable natural or recombinant proteins include, but are not limited to wheat and oat proteins, collagen, keratins, gelatin, elastin, fibronectin, soluble reticulin, soy proteins, chemically modified soy proteins, as described by Shultz et al. in U.S. patent application Ser. No. 10/868,332; PCT/US2004/019127 and water-soluble silk proteins, prepared as described by Fahnestock in U.S. Patent Application Publication No. 2004/0132978. The natural or recombinant proteins may be used in their native form or they may be modified to alter their ionic charge, for example by sulfonation or succinylation. These proteins may also be in partially hydrolyzed form by cleaving them into smaller peptide units.

The nail polish compositions of the invention may be prepared using conventional formulation and mixing techniques.

The invention also provides a method for coating nails with a nail polish film comprising a water or alcohol soluble polyamide comprising applying to the nails one of the nail polish compositions described above and allowing the composition to dry. The composition may be applied to the nails using any method known in the art. Typically, a standard brush applicator is used.

### EXAMPLES

The present invention is further defined in the following Examples. It should be understood that these Examples, while indicating preferred embodiments of the invention, are given by way of illustration only. From the above discussion and these Examples, one skilled in the art can ascertain the essential characteristics of this invention, and without departing from the spirit and scope thereof, can make various changes and modifications of the invention to adapt it to various uses and conditions.

All ingredients used in the preparation of the nail polish compositions described in the following Examples are available commercially unless otherwise noted.

The meaning of abbreviations used is as follows: “wt %” means percent by weight, “RV” means relative viscosity, “mL” means milliliter(s), “g” means gram(s), “psig” means pounds per square inch gauge, “kPa” means kilopascal(s), “min” means minute(s), “ppm” means parts per million, and “qs” means as much as suffices.

General Methods:

Preparation of the Polyamide Resins:

The polyamide polymerization was carried out using standard nylon polymerization processes that are well known in the art (See Kohan, M. I., Nylon Plastics Handbook, Hansen/Gardner Publications, Inc. [1995] pages 17-20 & 34-45). As is well known in the art, the stoichiometry of the ingredients was determined and controlled using pH measurements. The molecular weight during polymerization, as indicated by RV, was controlled by controlling pH, use of atmospheric nitrogen, or vacuum finishing after pressure reduction. Usually in polyamide polymerization, the molten polymer is quenched in water and then cut into pellets. However, because these polyamides are water-soluble, the molten polymer was either allowed to cool under ambient conditions or dropped onto a bed of ground dry ice for cooling.

Testing:

The RV in formic acid of an 8.4 wt % solution was determined at 25° C. using a Brookfeld Viscometer. The solubility in room temperature water (23° C.) at 10% concentration was determined by mixing 10 wt % of the polymer with 90 wt % demineralized water and stirring at room temperature. The solution was allowed to sit at room temperature, and the solution was observed for any sign of precipitation.

### Example 1

### Preparation of a Water-Soluble Polyamide

The purpose of this Example was to prepare a water-soluble polyamide by reacting triethyleneglycol diamine and adipic acid.

In a beaker provided with a stirrer, 300 mL of demineralized water and 444.0 g of triethyleneglycol diamine (TEGD) were mixed and heated to 60 to 70° C. with stirring. To the mixture was added slowly 438.0 g of adipic acid. An additional 100 mL of demineralized water was added. When all the adipic acid was dissolved, the pH was adjusted to 7.25 by addition of 7.2 g of TEGD. The solution was then introduced into a 3,785 mL autoclave where the solution was heated slowly until the pressure in the autoclave reached 250 psig (1724 kPa). At this point, steam was slowly vented while heating was continued. When the batch temperature reached 225° C., the steam venting was increased so as to lower the pressure to atmospheric pressure in 45 min, but at a rate such that the batch temperature continued to increase as it was being concentrated. The polymer was then subjected to a vacuum of 19.5 inches of mercury (66.0 kPa) for 60 min. At the end of 60 min, the batch temperature was 270° C. The autoclave was then pressured with nitrogen to force the polymer out of the autoclave and into a pan. The polymer was allowed to cool to room temperature. The properties of the water-soluble polyamide are given in Table 1.

### Examples 2-4

### Preparation of Water-Soluble Polyamides

The purpose of these Examples was to prepare water-soluble polyamides of different molecular weights by reacting triethyleneglycol diamine and adipic acid at different experimental conditions.

In a beaker provided with a stirrer, 1997.0 g of demineralized water and 740.0 g of TEGD were mixed with stirring. To the mixture was added slowly 730.0 g of adipic acid. When all the adipic acid was dissolved, 0.37 g of sodium hypophosphite monohydrate (SHP monohydrate) was added. The pH of the salt solution was 7.10. An 830.0 g portion of the salt was then introduced into a 3,785 mL autoclave where the solution was heated slowly until the pressure in the autoclave reached 250 psig (1724 kPa). At this point, steam was slowly vented while heating was continued. When the batch temperature reached 225° C., the steam venting was increased so as to lower the pressure to atmospheric pressure in 45 min, but at a rate such that the batch temperature continued to increase as it was being concentrated. The polymer was then held at atmospheric conditions for 20 min. At the end of 20 min, the batch temperature was 255° C. The autoclave was then pressured with nitrogen to force the polymer out of the autoclave and into a pan filled with ground dry ice.

The water-soluble polyamides of Example 3 and Example 4 were prepared using the same procedure as described above, with the exception that vacuum was used for the finishing step. The results are shown in Table 1.

TABLE 1
Properties of Water-Soluble Polyamides

| | | Catalyst¹ | | Water- |
|---|---|---|---|---|
| Example | Composition | (ppm) | RV | Solubility |
| 1 | TEGD, 6 | None | 12.9 | Soluble |
| 2 | TEGD, 6 | 210 | 14.0 | Soluble |
| 3 | TEGD, 6 | 349 | 20.5 | Soluble |
| 4 | TEGD, 6 | 210 | 22.8 | Soluble |

¹The catalyst was sodium hypophosphite monohydrate.

### Examples 5-8

### Preparation of Water-Soluble Copolyamides

The purpose of these Examples was to prepare water-soluble polyamides using caprolactam as comonomer.

In a beaker provided with a stirrer, 300 mL of demineralized water and 278.2 g of TEGD were mixed and heated to 60 to 70° C. with stirring. To the mixture was added slowly 274.5 g of adipic acid. When the adipic acid has dissolved, 269.0 g of caprolactam solution (81.86 wt %) was added. The pH was then adjusted to 7.35 by addition of 4.1 g of TEGD. The solution was then introduced into a 3,785 mL autoclave where the solution was heated slowly until the pressure in the autoclave reached 250 psig (1724 kPa). At this point, steam was slowly vented while heating was continued. When the batch temperature reached 225° C., the steam venting was increased so as to lower the pressure to atmospheric pressure in 45 min, but at a rate such that the batch temperature continued to increase as it was being concentrated. The polymer was then subjected to a vacuum of 22.0 inches of mercury (74.5 kPa) to 22.5 inches of mercury (76.2 kPa) for 60 min. At the end of 60 min, the batch temperature was 268° C. The autoclave was then pressured with nitrogen to force the polymer out of the autoclave and into a pan. The polymer was allowed to cool to room temperature.

The water-soluble copolyamides of Examples 6-8 were prepared using the same method, but with different ratios of comonomers, as shown in Table 2. The properties of the copolyamides are summarized in Table 2.

TABLE 2
Properties of Water-Soluble Copolyamides

| | | Mole | Catalyst¹ | | Water- |
|---|---|---|---|---|---|
| Example | Composition | Ratio | (ppm) | RV | Solubility |
| 5 | TEGD, 6/6 | 50/50 | None | 17.7 | Soluble |
| 6 | TEGD, 6/6 | 50/50 | 349 | 25.6 | Soluble² |
| 7 | TEGD, 6/6 | 70/30 | None | 15.3 | Soluble |
| 8 | TEGD, 6/6 | 80/20 | None | 14.1 | Soluble |

¹The catalyst was sodium hypophosphite monohydrate.
²Soluble, but dissolved much slower than Example 5.

### Examples 9-10

### Preparation of Alcohol-Soluble Copolyamides

The purpose of this Example was to prepare copolyamides that are alcohol-soluble using caprolactam or 2-methylpentamethylenediamine, 6 as comonomer.

The alcohol-soluble copolyamides were prepared using the method described in Examples 5-8 using the comonomers and ratios shown in Table 3. The resulting copolyamides had a lower water-solubility than that of the previous Examples, but are predicted to have a higher alcohol solubility.

TABLE 3
Properties of Alcohol-Soluble Copolyamides

| | | Mole | | |
|---|---|---|---|---|
| Example | Composition | Ratio | Catalyst | RV |
| 9 | TEGD, 6/6 | 40/60 | None | 19.0 |
| 10 | TEGD, 6/2MP | 65/35 | None | 17.7 |
| | MD, 6¹ | | | |

¹2-methylpentamethylenediamine, 6

### Examples 11-14

### Nail Polish Compositions

The purpose of these prophetic Examples is to describe the preparation of nail polish compositions containing a water or alcohol soluble polyamide.

Four nail polish compositions are prepared by mixing the following ingredients.

TABLE 4
Nail Polish Compositions

| | Example | Example | Example | Example |
|---|---|---|---|---|
| | 11 | 12 | 13 | 14 |
| Ingredients | (wt %) | (wt %) | (wt %) | (wt %) |
| Ethyl Acetate | 24.80 | 22.80 | 24.40 | 23.80 |
| Butyl Acetate | 20.70 | 19.70 | 20.70 | 20.70 |
| Propyl Acetate | 9.70 | 9.70 | 9.70 | 9.70 |
| Nitrocellulose | 14.00 | 14.00 | 14.00 | 14.00 |
| Isopropyl Alcohol | 1.03 | 1.03 | 1.03 | 1.03 |
| Butyl Alcohol | 9.00 | 9.00 | 9.00 | 9.00 |
| Diacetone Alcohol | 4.50 | 4.50 | 4.50 | 4.50 |
| Acrylates Copolymer | 4.40 | 4.40 | 4.40 | 4.40 |
| Triphenyl Phosphate | 4.57 | 4.57 | 4.57 | 4.57 |
| Tosylamide Epoxy | 2.40 | 2.40 | 2.40 | 2.40 |
| Resin | | | | |
| Water or Alcohol | 2.40 | 2.40 | 2.40 | 2.40 |
| Soluble Polyamide* | | | | |
| (10% in Isopropyl | | | | |
| Alcohol) | | | | |
| Acetyl Tributyl Citrate | 2.00 | 2.00 | 2.00 | 2.00 |
| Polyethylene | 0 | 3.00 | 0 | 0 |
| teraphthalate | | | | |
| D&C Red #6 Barium | 0 | 0 | 0.3 | 0 |
| Lake | | | | |
| D&C Red #34 Calcium | 0 | 0 | 0.1 | 0 |
| Lake | | | | |
| D&C Red #7 Calcium | 0 | 0 | 0 | 0.1 |
| Lake | | | | |
| Mica (Splendid Violet) | 0 | 0 | 0 | 0.7 |
| Ferric Ammonium | 0 | 0 | 0 | 0.02 |
| Ferrocyanide | | | | |
| Modified Urea- | 0.5 | 0.5 | 0.5 | 0.5 |
| Urethane (BYK410P) | | | | |

*The water or alcohol soluble polyamide is prepared as described in Example 9.

### Example 15

### Long Wearing Nail Enamel

The purpose of this prophetic Example is to describe the preparation of a long wearing nail enamel containing a water or alcohol soluble polyamide.

The long wearing nail enamel composition is prepared by mixing the following ingredients.

TABLE 5
Long Wearing Nail Enamel

| Ingredients | Wt % |
|---|---|
| Ethyl Acetate | 23.23 |
| Butyl Acetate | 22.31 |
| Nitrocellulose (10% in Isopropyl Alcohol) | 14.71 |
| Water or Alcohol Soluble Polyamide* | 11.21 |
| (10% in Isopropyl Alcohol) | |
| Dioctyl Adipate | 4.60 |
| Butylphthalimide Isopropylphthalimide | 3.73 |
| Sucrose Benzoate | 2.83 |
| Adhesion Promoter resin | 2.83 |
| UV Absorber | 0.15 |
| Citric Acid Solution | 0.20 |
| Stearalkonium Hectorite/Bentonite Gel | 14.20 |
| Total | 100 |

*The water or alcohol soluble polyamide is prepared as described in Example 10.

### Example 16

### Acrylic Nail Enamel

The purpose of this prophetic Example is to describe the preparation of an acrylic nail enamel containing a water or alcohol soluble polyamide.

The acrylic nail enamel composition is prepared by mixing the following ingredients.

TABLE 6
Acrylic Nail Enamel

| Ingredients | Wt % |
|---|---|
| Ethyl Acetate | 33.40 |
| PM Acetate | 3.000 |
| Nitrocellulose (70% in Isopropyl Alcohol) | 5.000 |
| Acrylates Copolymer | 25.00 |
| Acrylates/Dimethicone Copolymer | 3.000 |
| Dioctyl Adipate | 3.000 |
| Butylphthalimide Isopropylphthalimide | 3.600 |
| Water or Alcohol Soluble Polyamide* | 5.600 |
| (10% in Isopropyl Alcohol) | |
| UV Absorber | 0.200 |
| Citric Acid Solution | 0.200 |
| Stearalkonium Hectorite/Bentonite Gel | 18.00 |
| Total | 100 |

*The water or alcohol soluble polyamide is prepared as described in Example 9.

### Example 17

### Long Lasting Nail Enamel

The purpose of this prophetic Example is to describe the preparation of a long lasting nail enamel containing a water or alcohol soluble polyamide.

The long lasting nail enamel composition is prepared by mixing the following ingredients.

TABLE 7
Long Lasting Nail Enamel

| Ingredients | Wt % |
|---|---|
| Ethyl Acetate | 18.368 |
| Butyl Acetate | 3.772 |
| Isopropyl Alcohol | 2.979 |
| Butyl Alcohol | 1.490 |
| Nitrocellulose in Isopropyl Alcohol/ | 27.462 |
| Ester Solvents | |
| Water or Alcohol Soluble Polyamide* | 6.942 |
| (10% in Isopropyl Alcohol) | |
| Tosylamide/Epoxy Resin | 4.964 |
| Acrylates Copolymer | 2.483 |
| Stearalkonium Hectorite/Bentonite Gel | 13.910 |
| Fumed Silica Gel | 9.430 |
| Benzyl Benzoate | 2.500 |
| Butylphthalimide Isopropylphthalimide | 3.000 |
| Trioctyl Trimelliate | 2.500 |
| UV Absorber | 0.200 |
| Total | 100 |

*The water or alcohol soluble polyamide is prepared as described in Example 10.

### Examples 18-21

### Aqueous Nail Polish Compositions

The purpose of these prophetic Examples is to describe the preparation of aqueous nail polish compositions containing a water or alcohol soluble polyamide.

Four aqueous nail polish compositions are prepared by mixing the following ingredients.

TABLE 8
Aqueous Nail Polish Compositions

| | Example | Example | Example | Example |
|---|---|---|---|---|
| | 18 | 19 | 20 | 21 |
| Ingredient | (wt %) | (wt %) | (wt %) | (wt %) |
| Water or Alcohol | 40.00 | 30.00 | 40.00 | 30.00 |
| Soluble Polyamide* | | | | |
| (In Water 70%) | | | | |
| Zinc Stearate | 0.05 | 0 | 0.05 | 0 |
| Trioctyl Trimellitate | 0.05 | 0 | 0.05 | 0 |
| Rhodia V-06 | 1.5 | 0 | 1.5 | 0 |
| Silicone Adhesion | | | | |
| Butylphthalimide | 1.5 | 0 | 1.5 | 0 |
| Isopropyl Alcohol | 0 | 5 | 0 | 0 |
| Cosmetic Ferric | 1 | 1 | 1 | 1 |
| Ferrocyanide | | | | |
| (0.200 wt %) | | | | |
| D&C Red #6 | 1 | 1 | 1 | 1 |
| Barium Lake | | | | |
| (1.729 wt %) | | | | |
| D&C TiO₂ | 1 | 1 | 1 | 1 |
| (44.969 wt %) | | | | |
| Cosmetic Iron | 1 | 1 | 1 | 1 |
| Oxide M (3.216 wt %) | | | | |
| D&C Yellow #5 | 0 | 1 | 0 | 1 |
| Zirconium Lake | | | | |
| (1.366 wt %) | | | | |
| Isopropyl | qs to 100 | qs to 100 | 0 | 0 |
| Alcohol:Water | | | | |
| (50:50 Mixture) | | | | |
| Ethyl Alcohol | 0 | 0 | 0 | 5 |
| Ethyl Alcohol:Water | 0 | 0 | qs to 100 | qs to 100 |
| (50:50 Mixture) | | | | |

*The water or alcohol soluble polyamide is prepared as described in Example 5.

What is claimed is:

1. A nail polish composition comprising an effective amount of a water or alcohol soluble polyamide having a solubility of at least 0.5 weight percent in water or in a C₁ to C₃ alcohol and derived from the reaction of adipic acid and ether diamines with a molecular weight of 148 to 396 and represented by the general formulas: H₂N—R₁—O—R₂—O—R₁—NH₂, wherein R₁ and R₂ are either —CH₂—CH₂— or —CH₂—CH₂—CH₂—; or H₂N—R₁(—O—CH₂—CH₂)ₓO—R₁—NH₂, wherein R₁ is either —CH₂—CH₂— or —CH₂—CH₂—CH₂— and X has an average value of 2 to 6; and mixtures thereof.

2. The composition of claim 1 further comprising at least one cosmetic component selected from the group consisting of solvents, film-forming substances, plasticizers, dyes, pigments, iridescent substances, suspending agents, coalescing agents, thickeners, surfactants, wetting agents, slip aids, preservatives, vitamins, UV absorbing organic sunscreens, UV scattering inorganic sunscreens, natural proteins, and recombinant proteins.

3. The composition of claim 2 wherein the solvent is selected from the group consisting of acetone, ethyl acetate, butyl acetate, 2-methoxyethyl acetate, methyl ethyl ketone, methyl isobutyl ketone, methyl acetate, amyl acetate, isopropyl acetate, hexane, octane, toluene, xylene, ethanol, n-butanol, n-propanol, isopropanol, and mixtures thereof.

4. The composition of claim 2 wherein the solvent is water or a mixture of water and a water-miscible organic solvent selected from the group consisting of acetone, ethanol, n-butanol, n-propanol, isopropanol, and mixtures thereof.

5. The composition of claim 2 wherein the film-forming substance is selected from the group consisting of nitrocelluloses, polyvinyl derivatives, cellulose derivatives other than nitrocellulose, acrylic polymers, acrylic copolymers, acrylic resins, styrene resins, acrylate-styrene resins, vinyl resins, vinyl copolymers, polyester polymers, arylsulphonamide resins, alkyd resins, polyurethanes, polyurethane copolymers, and mixtures thereof.

6. The composition of claim 2 wherein the plasticizer is selected from the group consisting of tricresyl phosphate, benzyl benzoate, tributyl phosphate, butyl acetyl ricinoleate, triethyl citrate, tributyl acetyl citrate, dibutyl phthalate, camphor and mixtures thereof.

7. The composition of claim 2 wherein the surfactant is a water-soluble fluorinated surfactant.

8. The composition of claim 1 wherein the effective amount of water or alcohol soluble polyamide is from about 0.1% to about 40% by weight relative to the total weight of the composition.

9. The composition of claim 1 wherein the effective amount of water or alcohol soluble polyamide is from about 1% to about 40% by weight relative to the total weight of the composition.

10. The composition of claim 1 further comprising copolyamides of said water or alcohol soluble polyamide and one or more polyamide-forming comonomers.

11. The composition of claim 10 wherein said polyamide-forming comonomer is selected from the group consisting of polyethylene glycol diamine, polyethylene glycol diacids, and mixtures thereof.

12. The composition of claim 1 wherein the water or alcohol soluble polyamide is a copolymer with caprolactam and polyamides derived from hexamethylene diamine or 2-methylpentamethylene diamine and adipic acid or mixtures thereof.

13. The composition of claim 1 wherein the composition is in the form of a color enamel, nail varnish, nail lacquer, clear base coat, topcoat, or nail hardener.

14. A method for forming a coating of nail polish film comprising a water or alcohol soluble polyamide on nails comprising:
a) applying to the nails the composition of claim 1; and
b) allowing the composition to dry on the nails to form a film.
